(19) United States
(12) Patent Application Publication
(10) Pub. No.: US 2012/0142786 A1
(43) Pub. Date: Jun. 7, 2012

(54) OREGANO EXTRACT FOR ALERTNESS

(76) Inventors: Regina Goralczyk, Grenzach-Whylen (DE); Jonas Wittwer, Zuerich (CH); Hasan Mohajeri, Egg b.Zurich (CH)

(21) Appl. No.: 13/202,023

(22) PCT Filed: Feb. 19, 2010
(86) PCT No.: PCT/EP2010/052115

§ 371 Date: Oct. 31, 2011

Related U.S. Application Data
(60) Provisional application No. 61/154,023, filed on Feb. 20, 2009.
(60) Provisional application No. 61/222,213, filed on Jul. 1, 2009.

(51) Int. Cl. A61K 31/122; A61P 25/00; C07C 50/02; C07C 39/10; A61K 31/05; C07C 39/06

(52) U.S. Cl. 514/688; 514/731; 568/781; 552/309; 568/766

(57) ABSTRACT

Oregano extract can act as a stimulant, yet it does not interfere with sleep patterns or induce nervousness the way many stimulants such as caffeine can. It also has the benefits of promoting improved vigilance, improving attention and ability to focus on a task, and improving general alertness.

### FIELD OF THE INVENTION

This invention relates to the use of oregano extract and its active ingredients to increase a person's ability to stay focused, alert, and vigilant without the accompanying nervousness or agitation commonly experienced when taking stimulants (such as caffeine).

### BACKGROUND OF THE INVENTION

As people in Western society experience a longer lifespan, the need to remain independent will continue to grow. In order to stay independent and to ensure healthy living in later years, people have to remain healthy, both in body and in mind.

As we grow older, people often experience diminished attention, information processing speed, flexibility and short-term memory. However, even the non-elderly segment of the population can often experience similar memory problems when faced with stress and information overload due to life experiences such as starting a new job, overwhelming work deadlines, school competitions or exhausting and tiring social interactions.

Natural ingredients can be of help for people to naturally fight tiredness and to increase attention and vigilance. Among these natural ingredients, coffee is one of the most consumed. Coffee was shown to increase wakefulness and motor activity and to improve alertness and attention that can improve mental and work performance However, coffee has also side-effects depending on the consumer and the amount of coffee intake. Sensitive drinkers who consume more than a few cups of coffee at a time might experience insomnia, irritability, hand tremors, restlessness, nervousness, headaches, extra heartbeats and have a difficult time concentrating. Other side effects include a temporary rise in blood pressure, breathing rate and metabolism.

It would therefore be desirable to identify natural ingredients having the positive stimulating effects of coffee, but without the adverse side-effects.

### DETAILED DESCRIPTION OF THE INVENTION

It has been found, in accordance with this invention, and based on EEG and evoked potential testing, that administration of an oregano extract and/or its active ingredients results in a state of relaxation which is combined with vigilance, i.e., wakefulness, alertness, the ability to focus, and/or stimulates attention.

This one aspect of this invention is the use of oregano extract and/or its active ingredients to enhance vigilance, i.e. wakefulness, alertness, and focus in a subject. Another aspect of this invention is a food, nutraceutical, or pharmaceutical composition that enhances vigilance, i.e. wakefulness, alertness, and the ability to focus comprising an effective amount of oregano extract and/or its active ingredients. Another aspect is a method of enhancing vigilance comprising administering an effective amount of oregano to a subject and observing an enhanced vigilance.

Thus, one embodiment of this invention is a method of achieving a relaxed state while staying vigilant, i.e. wakeful, alert, and focused comprising ingesting an oregano extract in an amount sufficient to increase alpha-1 and beta-1 waves.

Another aspect of this invention is a method of increasing P300 peak amplitude comprising administration of oregano extract or its active ingredients.

Another aspect of this invention is the use of oregano extract as a food ingredient or food supplement so that after ingestion, a person will feel relaxed, yet alert, vigilant, focused, and attentive.

Another aspect of this invention is a method of improving vigilance comprising administering oregano extract, and observing enhanced vigilance. Another aspect of this invention is a nutraceutical, pharmaceutical or food or food comprising oregano extract or its active ingredients supplement which can induce wakefulness, yet not interfere with sleep patterns.

### BRIEF DESCRIPTION OF THE FIGURES

FIG. 1 shows Pharmaco-EEG inter-kinetic maps for absolute energy, alpha-1 and beta-1 waves, for oregano extract 120 mg compared with placebo. Significant changes are visible for alpha-1 and beta-1 waves after 1 hour. Significant ranges of positive changes in the direction of oregano extract are indicated according to the grey-scale.

FIG. 2 is the S300 mapping of integrated P300 response (statistically significant maps) showing significant positive changes (p<0.01) of P300 amplitudes for the 30 mg oregano extract dose between 2-6 hours after intake and significant positive changes (p<0.01) of amplitudes for the 60 mg oregano extract dose at 2 hours after intake compared to placebo.

FIG. 3 shows P300 peak amplitudes (in %) for the oregano extracts and placebo in relation to pre-dose (baseline) on the vertex lead (central electrode on the head). There is a significant increase of P300 amplitude with 60 mg oregano extract compared to placebo and compared with baseline 2 hours after intake (indicated with asterisk).

FIG. 4 shows P300 peak latency changes (in milliseconds) of oregano extract versus placebo. A trend of oregano extract to decrease P300 peak latency 1 hour after intake (10 ms reduction) can be observed.

FIG. 5 shows graphs of sleep profile parameters. None of the parameters were significantly changed.

The term “oregano extract and/or its active ingredients” is meant to include not only complete mixtures of extractable compounds but also only volatile components of the plant taken alone or in any combination with each other. The most important volatile components of oregano extracts in accordance with the present invention are: carvacrol, thymol, thymoquinone and thymoquinol. Thus, “oregano extract and/or its active ingredients” means that oregano extract, carvacrol, thymol, thymoquinone, thymoquinol or mixtures of two or more of the foregoing components may be present.

The expression “oregano extracts” of the present invention does not encompass teas or hot aqueous extracts made from fresh or dried leaves or any other parts of Oregano species, as teas will only contain trace amounts of the volatiles.

Extracts obtained by steam distillation are, however, in the scope of the present invention. Such extracts generally contain volatile compounds that are not readily degraded. Distilled oils contain hardly any thymoquinone and other volatiles, since they degrade more rapidly during steam distillation. However, they can contain high amounts of carvacrol. SFCO2 extracts (supercritical fluid carbon dioxide) are especially preferred for their stability (up to 5 years in closed containers).

The term “vigilance” encompasses one or more of the following traits: wakefulness, alertness, attentiveness, concentration and focus. Specifically, one's ability to increase and/or maintain concentration is enhanced, along with the ability to ignore surrounding signals not of relevance to the situation.

The term “observing enhanced vigilance” means that the observer may either be the person who ingests the active ingredients, or another observer. The observation may be a self-assessment, or may be based on objective measureable critieria.

### Assessment of Vigilance

Cognitive function and vigilance can be tested in several ways. Several cognitive tests have been developed and validated which measure different aspects of cognition such as spatial learning (cognitive development) or memory. However, these tests describe the outcomes of improved cognition, but do not focus on the neuro-biological activities. In addition, they often fail to identify small changes in cognitive performance. As an illustration, it is difficult to measure minor changes occurring in mild cognitive impairment. Further, questionnaires directed to assessing qualities such as vigilance, arousal, alertness and attention are generally not considered an accurate and robust measurement of these states. They are often subject to ethnic/cultural bias interpretation, or can be influenced by the level or quality of education.

Neuron-imaging techniques are one type of less-biased techniques which can give the opportunity to directly investigate the neuronal activity as a response to several stimuli or conditions in different brain areas. For assessing different levels of global brain activation, Electro Encephalograms (EEGs) and Event Related Potentials (ERPs) are the preferred methods because they reflect the temporo-spatial pattern of synchronized cortical neuronal mass activity and are the only noninvasive methods to measure neuronal activity directly and with a sufficient time resolution.

The EEG measures ongoing electrical activity generated by the neurons in the brain resulting in brainwaves of different frequencies. These waves can be divided into several frequency bands, in particular Delta (0.5- 3.5 Hz), Theta (4- 7.5 Hz), Alpha (8- 12.5 Hz), and Beta (13- 32 Hz). These waves reflect the state of brain function that the person experiences at the moment of the recording:
delta waves normally occur in deep sleep;
theta waves are seen in connection with intuition, daydreaming and fantasizing and therefore reflect the state between wakefulness and sleep;
alpha waves reflect a state of relaxation and alertness and are the brain's most important waves associated with learning and using information; and
beta waves are associated with mental activity, alertness, problem solving, judgment, decision making and processing information, all components of “vigilance”.

One measurement of ERPs is the “P300” peak. This peak is an evoked potential (e.g., by visual or acoustic stimulus) referred to as a “cognitive” or “event-related response” occurring in the 300 msec latency region with a large positive voltage peak. Attention and state of arousal are the two most important factors in eliciting a P300 response. P300 amplitudes and latencies are used clinically to assess patients with Alzheimer's Disease, Parkinson's Disease, and dementia. Patients with these neurodegenerative disorders tend to have prolonged P300 latencies, believed to be related to changes in neurotransmitters. P300 latencies have been shown to increase (while amplitudes decrease), with decreases in cognitive function.

It has been found, it accordance with this invention that oregano extract and its active ingredients are able to significantly increase alpha-1 and beta-1 EEG parameters in the resting condition.

An increase in alpha activity has been associated with relaxation, increased creativity, increased performance under stress and improved learning and concentration, as well as decreased anxiety. (Eschenauer et al 2006 Am J Syst Pharm 63:26-30; Boutcher et al 1988 Physcophysiology 25: 696-702). An increase in beta activity is related to higher cortical activation (Kubitz et al 1996 Res Q Exerc Sport 67: 91-96), an increased state of alertness (Porjesz et al 2002 BiolPhychol 61:229-48) and cognitive processing (Karrasch et al 2004 Neurosci Lett 366:18-23).

Thus, in accordance with this invention, oregano extract or its active ingredients can improve relaxation, increase creativity, increase performance under stress, improve concentration, and decrease anxiety as it increases alpha-1 waves. By increasing beta-1 waves, oregano extract or its active ingredients can increase alertness, concentration and thus increase vigilance.

Also it has been shown in accordance with this invention, that oregano extract and its active ingredients can significantly increase the surface P300 peak amplitudes (S300) as well as the P300 peak amplitudes on the vertex lead. The oregano extract also decreases the P300 peak latencies.

An increase of P300 peak amplitude has been related to a higher amount of selective attention (van Nunen et al 1994 Acta Phychiatr Belg 94: 96-97), whereas shorter P300 peak latencies have been related to superior cognitive performance by a faster processing time for the discrimination, referencing, and evaluation of stimuli (van Nunen, supra; Hansenne 2000 Neurophysiol Clin 30: 191-210; Emmerson et al 1989 Exp. Aging Res 15:151-9)

Another aspect of this invention is the use of oregano extract to compensate for the daily circadian decline in attention, wakefulness and alertness (vigilance) e.g., before and after lunch (mid day). It was also found, in accordance with this invention, that the subjects who received oregano extract did not experience the decrease of attention around midday as is often felt by many people. According to our experiments, the placebo group experienced a decrease of selective attention, alertness, wakefulness and focus on their assigned tasks towards lunchtime as measured by a reduction of P300 peak amplitudes on the vertex lead. However, in the group receiving the oregano extract, this was not observed. The subjects receiving oregano extract showed an increase of attention of 7% compared to the attention in the early morning.

A circadian decline of attention towards midday has also been shown by Kirkcaldy 1984 Eur J Appl Physiol Occup Physiol 52: 375-9. In the Kirkcaldy study, subjects reported to feel less activated and less alert around midday (between 11:00 AM and about 2:00 PM). Thus, another aspect of this invention is a method of avoiding a decrease of attention and/or alertness at midday by administering an effective amount of oregano extract between 11:00 AM and 2:00PM, or not later than one hour after a lunch time meal.

Therefore, these results indicate that oregano extract supports a state of relaxation, which is combined with wakefulness, alertness and focus.

Dosages Dosages for an adult of average body weight range from 25-200 mg oregano extract or its active ingredients per day; preferably from about 30-180 mg per day. The dosage can be adjusted if required. For a nutritional supplement, the dosage may be in the form of a capsule, a tablet, sachets, or any other conventional dosing form as is known in the art.

For optimal vigilance and alertness it is recommended that an adult take a single 60mg dosage in the morning. Conveniently, the dosage form may be in the form of a capsule. A second 60mg capsule (or other dosage form, such as a tablet) can be taken at or shortly after lunch, to prevent post lunch decline in the afternoon. Alternatively, one 120mg capsule can be taken once per day, preferably in the morning. In an alternative embodiment, a 30mg dose can be taken 3 times a day, i.e. in the morning at lunch and in the late afternoon, if a person wishes to extend their wakefulness in the evening. This application will not cause any sleeplessness in the night.

Alternatively, the dosage may be in the form of a functional food, where the oregano extract is added to various foodstuffs, including beverages.

Suitable oregano extracts are commercially available. One preferred extract is an SFCO2 extract available from Flavex Gmbh, Rehlingen, Germany.

The following non-limiting examples are presented to better illustrate the invention.

### EXAMPLE 1

### Assessing Effects of Oregano Extract on Neuronal Function by Quantitative Wake and Sleep EEG and Wake ERP in a Human Clinical Trial

In a single center, randomized, placebo controlled, cross-over human clinical trail on 20 healthy young male volunteers, the effects of three different doses of oregano extract (purchased from Flavex Gmbh, Rehlingen, Germany) (30 mg/60 mg/120 mg) on brain activity in a wake situation was tested against placebo with the method of quantitative Electro-Encephalogram (qEEG) and Event-Related Potentials (ERPs). In addition, polysomnographic recordings and all night sleep EEG spectral analysis in the 20 volunteers was performed in order to test the effect of oregano extract on sleep after an additional dose of 30 mg, 60 mg or 120 mg of oregano extract.

### Methods:

The 4 successive assessment periods were at least 5 days apart of each other (wash-out periods). At these periods, each subject received one of the 3 doses tested (30 mg, 60 mg or 120 mg) or the placebo as a single assessment. Three subjects did not complete the entire study periods.

At each study day of each period, qEEGs were recorded after dose administrations. In detail: 28 EEG leads were recorded using ear linked references as well as 4 artifact channels (detection of eye movement, muscle activity and other potentials causes of artifacts). EEGs were taken under first 3 minutes vigilance controlled recording (VC) conditions (the subjects are asked to push two knobs during the recording conditions), followed by 3 minutes resting (R) recording conditions (subjects are asked to relax with their eyes closed).

A double baseline was performed at 1 and 0.5 hours before dosing. Only the second baseline was used for analysis, the first one being a training session. Additional qEEG measurements were performed 1, 2, 4 and 6 hours after dosing. Extraction of parameters was carried out on individual spectra by breaking them down into standard frequency EEG bands: Delta (0.5-3.5 Hz), Theta (4-7.5 Hz), Alpha (8-12.5 Hz), and Beta (13-32 Hz). The alpha and the beta bands are also respectively divided in: Alpha 1 (8-9.5 Hz) and alpha 2 (10-12.5 Hz) and beta 1 (13-17.5 Hz), beta 2 (18-20.5 Hz) and beta 3 (21-32 Hz).

In addition, ERP measurements were performed. This ERP was based on the standard auditory P300 “odd-ball” paradigm. Each subject listened to a series of two tones, with a frequency of 500 Hz for frequent tones and a frequency of 2000 Hz for infrequent or target tones. Subjects were asked to count the infrequent tones. By this counting, a peak appeared in the EEG after about 300 ms. From this peak, the P300 amplitude, latency on Cz (central) electrode as well as 5300 (area under the P300 waveform) on all electrodes can be determined. Auditory P300 measurement time points were the same as for qEEG.

In the evening of each study day, the study volunteers got a second dose of the oregano extract (30 mg/ 60 mg/ 120 mg) or placebo and underwent polynomnography recording (sleep EEG) two hours later for the whole night (8 hours). Each treatment night was preceded by a habituation night that was not analyzed due to the first-night effect issue, i.e. difficulties in initiating and/or maintaining sleep that healthy subjects generally experience during a first recorded night in an unusual setting. Sleep stages were visually scored for the complete recording period (from 11:00 p.m. to 7:00 a.m.) at 30 sec intervals in either stage 0 (wake), stage 1, stage 2, stage 3, stage 4, stage 5 (rapid eye movement [REM] sleep) or stage 6 (movement time). The different visual sleep parameters were derived from the visual scoring of the recordings using the Hypnos software.

### Results

### EEG:

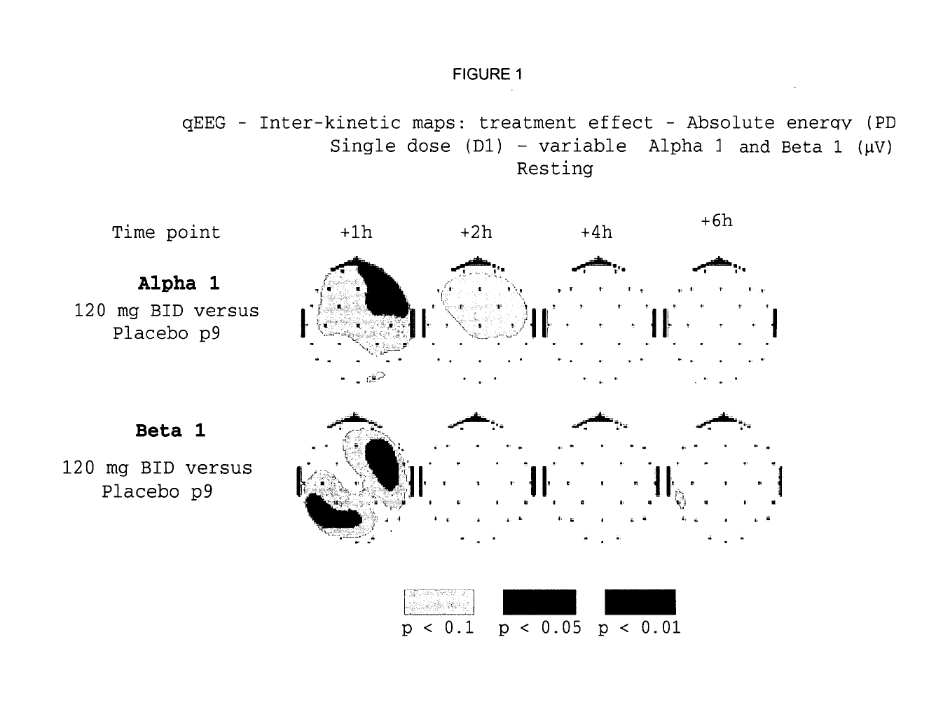

Quantified EEG recording and analysis was followed by a systematic evaluation method to search for compound-induced effects. These analyses revealed that in the wake qEEG, transient significant increases (p<0.05) in absolute power in the resting condition of alpha-1 and beta-1 EEG parameters have occurred for the 120 mg dose lasting for more than one hour (FIG. 1). Also at two hours after intake of the 120 mg dose of oregano extract, the alpha-1 wave was increased in comparison to placebo, although not statistically significant anymore (p<0.1).

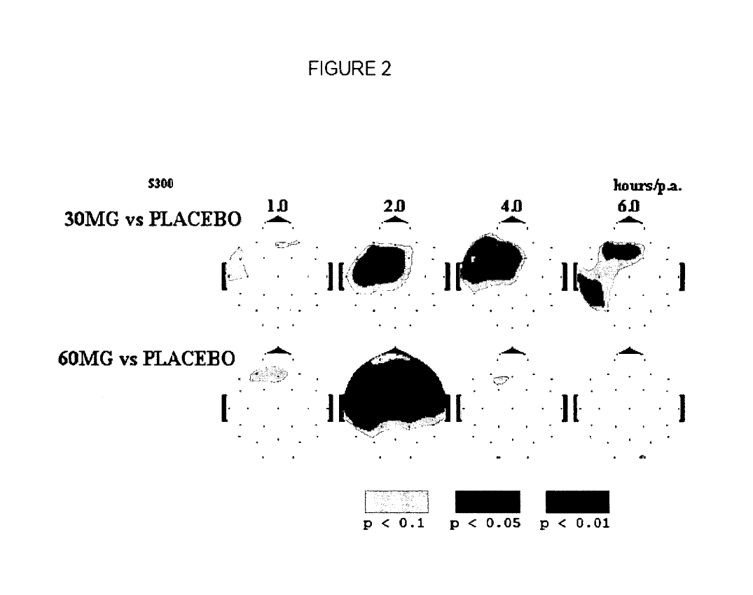

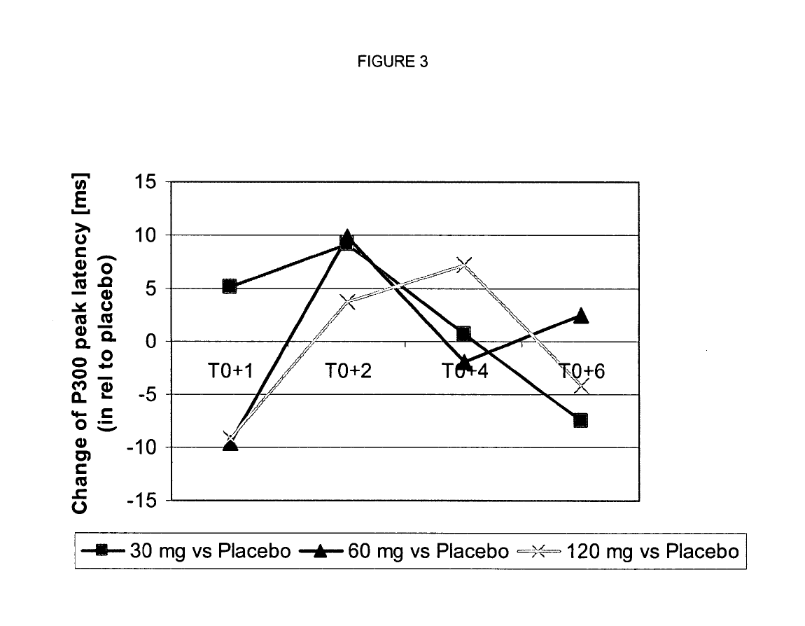

ERP: Analyses of the P300 peak latency and amplitude on the vertex lead showed some significant modifications by the oregano extract in comparison to placebo. Most evident were the increases in P300 amplitude maps for the 30 mg oregano extract dose over left frontal and central scalp regions, but most pronounced for the 60 mg oregano extract dose over the anterior half of the scalp compared with placebo. These modifications started to occur between 1-2 hours after oregano extract intake and lasted up to 6 hours in the case of the 30 mg dose and up to 2-3 hours for the 60 mg dose (FIG. 2). In addition, the P300 peak latencies were decreased one hour after intake of the 60 mg and the 120 mg oregano extract dose compared with placebo (not statistically significant, but a trend is observed) (FIG. 3).

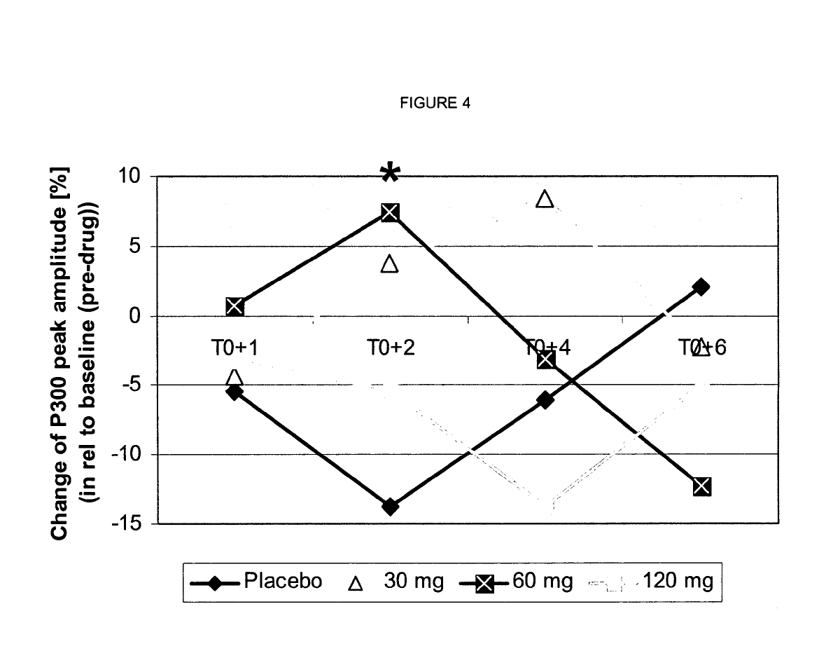

When looking solely at the P300 peak amplitudes on the vertex lead (central lead on the scalp), 60 mg oregano extract significantly increased the P300 amplitude by 7.4% compared with baseline, and 21.2% compared with placebo after 2 hours intake (FIG. 4).

The 30 mg dose increased the P300 amplitude for up to 8% compared with baseline and 15% compared with placebo after four hours of intake, but this was not statistically significant. Interestingly, the amplitudes in the placebo group were decreased 2 hours after placebo intake, which resembles a circadian decline in attention (before and after lunch). The 60 mg (and to a certain extent also the 30 mg) oregano extracts were able to alleviate this decline and to even increase the P300 amplitudes to higher levels than at baseline.

Therefore, the obtained results with the oregano extract indicate that oregano extract stimulates attention and helps to alleviate the daily decline in attention usually experienced at mid-day.

### Sleep EEG:

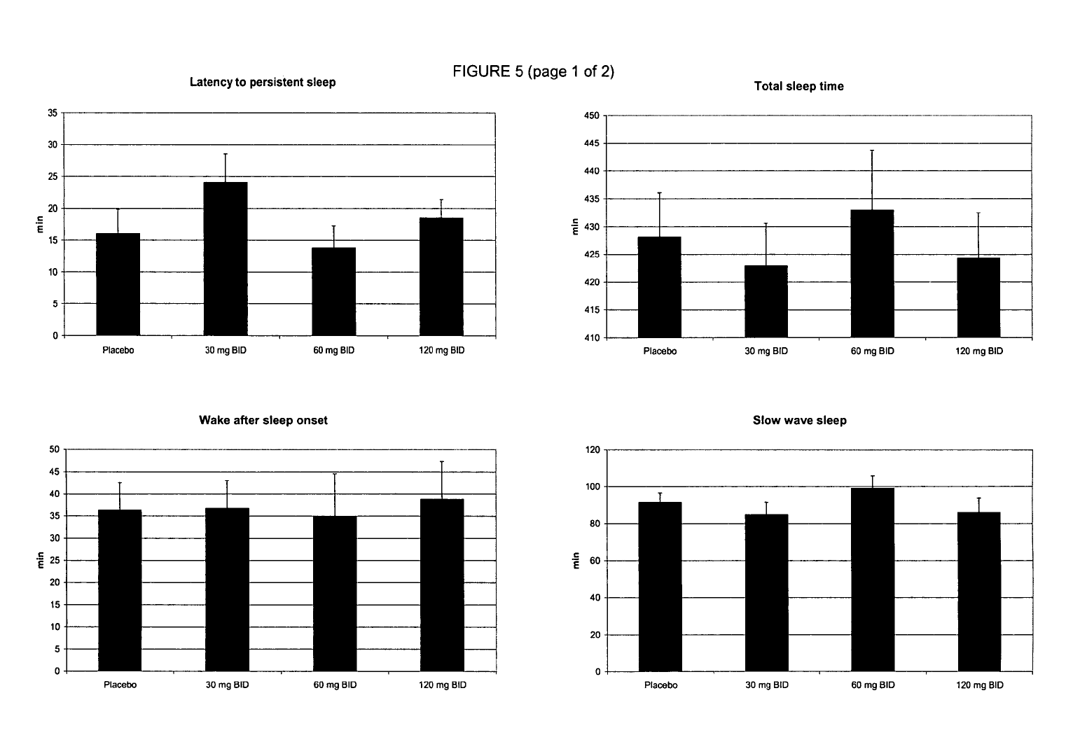

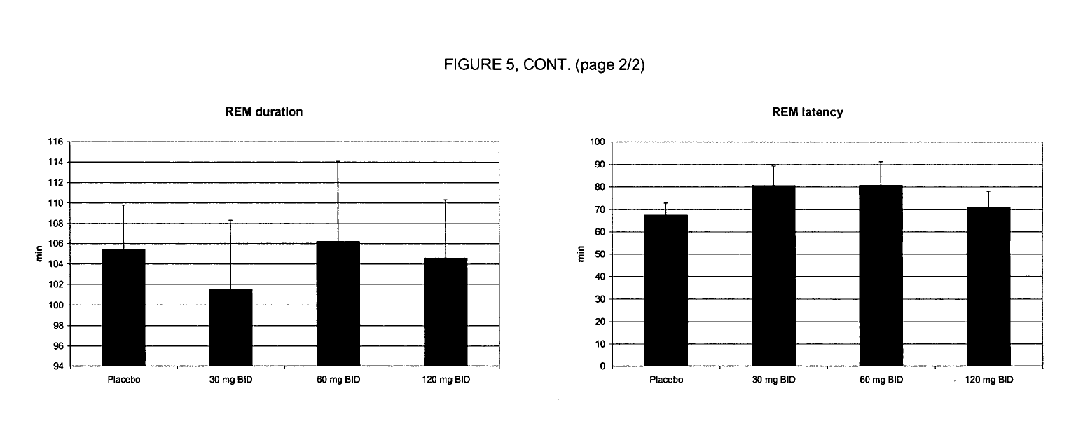

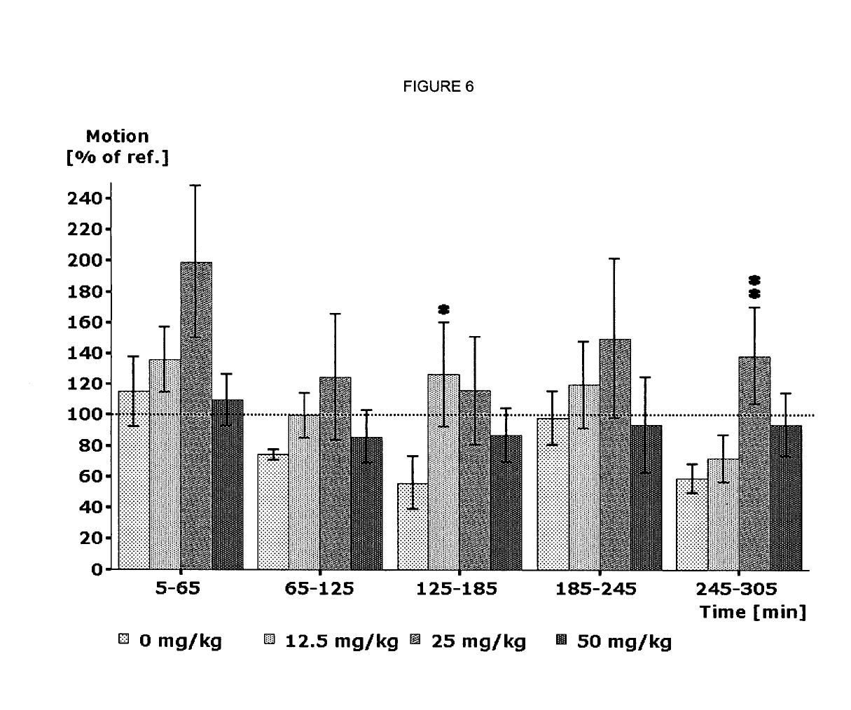

In order to test if the oregano extract influences sleep continuity or architecture, polysomnography recordings were done. Polysmnopgraphy data recording started two hours after the second intake of the study compound at the study day. After analyzing the entire set of sleep parameter data of all subjects, no deleterious effects on sleep continuity or architecture were observed (FIG. 5).

It has been found, in accordance with this invention that oregano extract and its active ingredients are able to significantly increase alpha-1 and beta-1 EEG parameters (absolute energy) in the resting condition of alpha-1 and beta-1 EEG parameters with a dose of 120 mg for up to two hours. Also the P300 surface peak amplitude (S300) was significantly increased for the 30 and 60 mg dose, occurring after one hour and returning to baseline level after 6 hours. Further, P300 peak amplitudes on the vertex lead were significantly increased 2 hours after 60 mg oregano extract administration in comparison to the placebo (+22%). Also compared with baseline, the 60 mg dose was significantly increasing the P300 amplitude (+7%) on the vertex lead after two hours. In addition, the P300 peak latencies showed a trend to be decreased one hour after intake of the 60 mg and the 120 mg oregano extract dose compared with placebo (−10ms).

An increase in alpha activity has been associated with relaxation, increased creativity, increased performance under stress and improved learning and concentration, as well as decreased anxiety ^{5,6}. An increase in beta activity is related to higher cortical activation ⁸, an increased state of alertness⁷ and cognitive processing ⁹.

### EXAMPLE 2

### Preparation of Liquid Capsules (LiCaps)

Liquid capsules (LiCaps) may be prepared comprising the following ingredients:

| Oregano extract Dosage | 30 mg | 60 mg | 120 mg |
|---|---|---|---|
| Oregano extract per capsule (mg) | 34.5 mg | 69 mg | 138 |
| Tricylglycerol (Durkex 200) (mg) | 420 | 420 | 420 |
| Phosphatidylcholine (mg) | 265.5 | 231 | 162 |
| Total per capsule (mg) | 720 | 720 | 720 |

### REFERENCES

1. Davis, M. & Whalen, P. J. The amygdala: vigilance and emotion. Mol Psychiatry 6, 13-34 (2001).
2. Hegerl, U. et al. EEG-vigilance differences between patients with borderline personality disorder, patients with obsessive-compulsive disorder and healthy controls. Eur Arch Psychiatry Clin Neurosci 258, 137-43 (2008).
3. Klerman, E .B., Boulos, Z., Edgar, D. M., Mistlberger, R. E. & Moore-Ede, M. C. EEG delta activity during undisturbed sleep in the squirrel monkey. Sleep Res Online 3, 113-9 (2000).
4. Bodizs, R., Sverteczki, M. & Meszaros, E. Wakefulness-sleep transition: emerging electroencephalographic similarities with the rapid eye movement phase. Brain Res Bull 76, 85-9 (2008).
5. Eschenauer, G. & Sweet, B. V. Pharmacology and therapeutic uses of theanine. Am J Health Syst Pharm 63, 26, 28-30 (2006).
6. Boutcher, S. H. & Landers, D. M. The effects of vigorous exercise on anxiety, heart rate, and alpha activity of runners and nonrunners. Psychophysiology 25, 696-702 (1988).
7. Porjesz, B. et al. Linkage and linkage disequilibrium mapping of ERP and EEG phenotypes. Biol Psychol 61, 229-48 (2002).
8. Kubitz, K. A. & Mott, A. A. EEG power spectral densities during and after cycle ergometer exercise. Res Q Exerc Sport 67, 91-6 (1996).
9. Karrasch, M., Laine, M., Rapinoja, P. & Krause, C. M. Effects of normal aging on event-related desynchronization/synchronization during a memory task in humans. Neurosci Lett 366, 18-23 (2004).
10. Kugler, C. F., Taghavy, A. & Platt, D. The event-related P300 potential analysis of cognitive human brain aging: a review. Gerontology 39, 280-303 (1993).
11. Polich, J. & Herbst, K. L. P300 as a clinical assay: rationale, evaluation, and findings. Int J Psychophysiol 38, 3-19 (2000).
12. Polich, J., Howard, L. & Starr, A. P300 latency correlates with digit span. Psychophysiology 20, 665-9 (1983).
13. Schellenberg, R., Sauer, S. & Dimpfel, W. Pharmacodynamic effects of two different hypericum extracts in healthy volunteers measured by quantitative EEG. Phannacopsychiatry 31 Suppl 1, 44-53 (1998).
14. Rechtschaffen, A. & Kales, A. A manual of standardized terminology techniques and scoring system for sleep stage of human subjects. Washington D.C.: National Institute of Health Publication 204(1968).
15. van Nunen, P.E. & Declerck, A. C. P300, alertness and cognition. Acta Psychiatr Belg 94, 96-7 (1994).
16. Hansenne, M. [The p300 cognitive event-related potential. I. Theoretical and psychobiologic perspectives]. Neurophysiol Clin 30, 191-210 (2000).
17. Emmerson, R. Y., Dustman, R. E., Shearer, D. E. & Turner, C. W. P3 latency and symbol digit performance correlations in aging. Exp Aging Res 15, 151-9 (1989).

1. A method of increasing alpha-1 and/or beta-1 brain wave activity comprising ingesting an effective amount of oregano extract or its active ingredients.

2. A method according to claim 1 wherein the active ingredients are selected from the group consisting of carvacrol, thymol, thymoquinone, thymoquinol, and mixtures of two or more of the foregoing.

3. A method according to claim 1 wherein the alpha-1 and/or brain wave activity is observed by achieving a relaxed state while staying wakeful, alert, vigilant or focused.

4. A method of improving attention or vigilance while not interfering with sleep patterns comprising ingesting an effective amount of oregano extract or its active ingredients.

5. A method of increasing evoked potential P300 peak amplitude comprising administration to a subject of an effective amount of oregano extract or its active ingredients.

6. A method according to claim 5 wherein the active ingredients are selected from the group consisting of carvacrol, thymol, thymoquinone, thymoquinol, and mixtures of two or more of the foregoing.

7. A method according to claim 6 wherein the P300 peak amplitude results in the subject achieving relaxed state while staying wakeful, alert, vigilant or focused.

8. A method according to claim 1 wherein the oregano extract of its active is ingested between 11:00 AM and 2:00 PM

9. Use of oregano extract or its active ingredients to increase alpha-1 and/or beta-1 brain wave activity.

10. Use according to claim 9 wherein the active ingredients are selected from the group consisting of carvacrol, thymol, thymoquinone, thymoquinol, and mixtures of two or more of the foregoing.

11. Use according to claim 9 wherein the alpha-1 and/or brain wave activity is observed by achieving a relaxed state while staying wakeful, alert, vigilant or focused.

12. Use according to claim 9 improve attention or vigilance while not interfering with sleep patterns.

13. Use according to claim 9 to increase evoked potential P300 peak amplitude.

14. Use according to claim 13 wherein the P300 peak amplitude results in the subject achieving relaxed state while staying wakeful, alert, vigilant or focused.

15. Use according to claim 9 where the use is between 11:00 AM and 2:00 PM.

16. A nutraceutical, food, or food supplement comprising oregano extract or its active ingredients, which when ingested, promotes vigilance in a person.
